(19) United States
(12) Patent Application Publication
(10) Pub. No.: US 2007/0250343 A1
(43) Pub. Date: Oct. 25, 2007

(54) Medical services and goods exchange

(76) Inventor: Ravinder Sohal, Palo Alto, CA (US)

Correspondence Address: FLIESLER MEYER LLP, 650 CALIFORNIA STREET, 14TH FLOOR, SAN FRANCISCO, CA, 94108, US

(21) Appl. No.: 11/446,866

(22) Filed: Jun. 5, 2006

Related U.S. Application Data
(60) Provisional application No. 60/793,771, filed on Apr. 21, 2006.

(51) Int. Cl. G06Q 10/00; A61B 5/00

(52) U.S. Cl. 705/2; 600/300

(57) ABSTRACT

Computer implemented methods and systems for reducing costs for medical goods and services. In an embodiment, a host system maintains service provider information, which includes, for each of a plurality of medical imaging services providers within the payor's network, a list of medical imaging services that are provided by the service provider and a corresponding fee charged for each medical imaging service. In response to receiving an order, from a referrer, for a recommended medical imaging service for a patient, the system identifies, based on the service provider information, service providers capable of performing the recommended medical imaging service. The system also selects at least one service provider capable of performing the recommended medical imaging service. Where more than one service provider is selected, a patient can be given the option of making a final selection of which service provider will perform the medical imaging service for the patient.

### PRIORITY CLAIM

This application claims priority under 35 U.S.C. 119(e) to U.S. Provisional Patent Application No. 60/793,771, which was filed Apr. 21, 2006.

### CROSS REFERENCE TO RELATED APPLICATION

This application is related to the U.S. patent application Ser. No. ______, entitled “SYSTEMS AND METHODS FOR AUTOMATICALLY GENERATING BIDS FOR MEDICAL SERVICES AND GOODS” (Atty. Docket No.: SOHAL-01000US2), which was filed the same day as the present application, and which is incorporated herein by reference.

### FIELD OF THE INVENTION

Embodiments of the present invention relate to systems and methods for reducing the costs for obtaining medical services and goods. Specific embodiments relate to systems and methods for reducing the costs of medical imaging services, while maintaining and preferably increasing the quality of such medical imaging services.

### BACKGROUND

Medical imaging services include both the acquisition of medical images and the interpretation of such images. Medical images can be created using many different types of imaging modalities, including, but not limited to, x-ray, computed radiography, magnetic resonance imaging MRI), computed tomography (CT), ultrasound imaging, nuclear medicine, and mammography, etc. Each type of modality requires a different type of imaging equipment. Additionally, for a specific modality there can be more than one type of equipment for obtaining the medical image. For example, an MI image can be acquired using a standard closed tube MRI scanner, or an open bore MRI scanner. Further, there are many different models of each type of equipment, with certain models being superior to others, and newer models typically incorporating improvements over older models.

There are various types of facilities, such as, but not limited to, hospitals, doctor's offices, free-standing imaging centers, or mobile imaging units (e.g., ultrasound vans, MRI/CT/PET trucks) that have the medical imaging equipment and the personnel to operate the equipment. Such facilities may also employ or be associated with practitioners that interpret the acquired medical images. In some instances the personnel that operate the imaging equipment to acquire medical images is the same as the personnel that interprets the images. In other instances, the personnel that acquire the images and interpret the images are different. It is also possible that medical images acquired at a facility are interpreted by personnel that are not located at and/or associated with the imaging facility. Further, depending on a patient's condition, it may be preferred that a specific type of specialist interpret the patient's medical images. For example, it may be preferred that a Neuroradiologist interpret an MRI of a patient's brain to look for a tumor, while it is preferred that a different type of radiologist interpret an MRI of a patient's shoulder to look for a subtle muscle tear.

It has been estimated that medical imaging costs are in the range of $100 billion per year. It has also been projected that such costs will continue to increase as the population ages, causing larger portions of the population to be candidates for medical imaging. Additionally, advances in medical imaging technology will also result in increased costs.

Quite often third party payors, such as, but not limited to health insurance providers, managed care organizations and the government (e.g., through Medicaid/Medicare or veteran's benefits), are responsible for paying at least a portion (and usually a significant portion) of the costs associated with the above mentioned medical imaging services. Due to the high costs and complexity associated with these medical imaging services, provider-sponsored networks (PSNs) and commercial radiology benefits management (RBM) organizations have been established to try to mange the utilization and costs of imaging.

RBM companies currently claim to cover over 50 million lives nationwide and involve hundreds of thousands referring physicians. Economic savings are accomplished by RBM companies through an emphasis on more appropriate ordering of high tech medical imaging exams, with the major RBM companies relying on some degree of direct intervention between the referring physician and the payor. This is accomplished through call centers, peer-to-peer consultation, web based authorization programs, and physician education. Additionally, many of the RBM companies have developed “steering” programs in which the RBM companies have contracted with low cost imaging providers and created local networks of radiology centers to which they can direct their clients' patients.

RBM companies are well established and the payor industry has accepted their methodology of addressing the issue of managing medical imaging costs. RBM companies assert that their programs deny 10 to 25 percent of all medical imaging requests, and that 40 percent of all insurers use some form of RBM. Some RBM companies also say that they obtain significant savings through steering programs. On average, RBM companies claim that they save about $1 per plan member per month (PMPM).

The RBM industry faces many challenges as high tech medical imaging services continue to grow. As the number of ordered medical imaging exams rises, RBM companies will need to invest in a highly compensated large screening pool of licensed professionals. They will also need to invest in large call centers and in technology that refines their screening processes.

The RBM industry has not developed a mutually beneficial relationship with medical imaging providers. This is because many steering programs are based on contracted, fixed “take it or leave it” low rates with local medical imaging providers in the payor's coverage area. Such fixed rates are sometimes so low that local imaging providers opt not to join the RBM network, or decide to leave the network, thereby limiting the imaging capacity for the payor.

Referring physicians are often wary of utilization measures. These physicians are likely to apply pressure to the payors if there is continued and increased utilization control. Physicians report that they are often required to “educate” screeners, which is time consuming, and thus not time or cost efficient. Further, physician office staff members frequency spend significant time relaying supporting clinical documentation to call centers, which is also costly and time consuming.

RBM companies measure their success through growth suppression. Although the first year of utilization review may rein in growth to nearly 0%, the RBM companies see their growth suppression bounce back to nearly half of the unmanaged level by the second year and overall imaging costs rise again.

As described below, there have been various attempts to reduce the costs associated with medical services. Some of these attempts have specifically related to reducing the costs associated with medical imaging services, while others are much more general, and are typically not applicable to medical imaging services.

For example, U.S. Pat. No. 6,006,191 to DiRinzo, which is incorporated herein by reference, attempts to reduce the costs associated with interpreting medical images through use of a bidding system where patients and physicians can negotiate a price for the physician's services. In this system, already acquired medical images are stored electronically so that they can be accessed by physician's at geographically dispersed locations. Patients provide bids that specify the amount that they are willing to pay to have their images interpreted. Physicians that are available to interpret such images can then search through the bids, decide whether to accept a bid, and thereafter download the images associated with the bid and interpret the images. A disadvantage of this system is that it does not provide any means for a third party payor (e.g., a health insurance provider) to participate in the bidding process. In fact, it purposefully leaves such third party payors out of the decision process. Another disadvantage of this system is that it does not address how to reduce the costs associated with acquiring the medical images in the first place.

U.S. Patent Application Publication No. 2005/0065821, to Kalies, Jr., which is incorporated herein by reference, discusses a reverse auction that enables pre-qualified prescription providers to bid to fulfill prescriptions. In this system, a bidding service provider supplies unfilled prescription information to a registry of pharmacies that have been previously qualified to participate in the bidding process. The customer can then select one of the bids or decline all bids. Selection can be based on idiosyncratic criteria such as a preferred pharmacy, cost, ancillary service offered, proximity of responding pharmacies to the customer, and so on. A disadvantage of the system of this publication is that does not provide any means for a third party payor (e.g., a health insurance provider) to participate in the bidding process, other than allowing the third party payor to set minimum prices for drugs (to maintain marketplace competition), and allowing the third party payor to authorize a prescription or ancillary service included in a bid. More specifically, the third party payor does not have a direct role in selecting the winning bid, or in selecting which bids a patient may select from. Additionally, while this publication says that its bidding system can be used for administration of medical treatments or services, it does not address how it can be used in the complex field of medical imaging services.

PCT Publication WO 00/72207, which is incorporated herein by reference, discusses a system where users can post a request for proposal (RFP), which is made available to service provides selected by the user. A disadvantage of the system of this publication is that does not provide any means for a third party payor (e.g., a health insurance provider) to participate in the bidding process. Additionally, while this publication says that its bidding system can be used to receive bids for medical services, it does not address how it can be used in the complex field of medical imaging services.

U.S. Patent Application Publication No. 2003/0195838 to Henley, which is incorporated herein by reference, describes a system for supporting an on-line auction for medical services. According to the publication, the system provides a mechanism whereby an insurance company can identify an underutilized facility to negotiate a lower price for a policy holder. However, in this system it is the third party payor or the patient that is bidding on medical services, which can have the effect of maximizing profits for the medical service provider, as opposed to reducing costs for the third party payors (and reducing the costs for patients by reducing the patients' insurance premiums, co-payments and/or percentage payments).

Accordingly, there is a still a need for new methods and systems for reducing the costs to third party payors, such as insurance companies, for providing medical imaging services. Preferably, such methods and systems should not alienate referring physicians and should not adversely affecting patient care. Preferably, such methods should provide for improved patient care and satisfaction.

### SUMMARY

Embodiments of the present invention are directed to computer implemented systems and methods for reducing costs for medical imaging services in a supply chain that includes a referrer (e.g., a doctor), a patient, a medical imaging service provider (e.g., an imaging center) and a payor (e.g., an insurance company) that is responsible for paying at least a part of the fee charged by the service provider for the medical imaging service. In accordance with an embodiment, a system of the present invention maintains service provider information (e.g., in a database), that includes, for each of a plurality of medical imaging services providers within a payor's network, a list of medical imaging services that are provided by the service provider and a corresponding fee charged for each of the medical imaging services. Such a list of medical imaging services included for each of the service providers can include commonly used procedure descriptors, such as but not limited to Current Procedure Terminology (CPT) codes, and the fee charged by the service provider for the medical imaging service corresponding to each procedure descriptor.

The system can also allow the service providers to change their corresponding service provider information, including the fees they charge. In accordance with an embodiment, the system allows the payor to specify a maximum fee that can be charged for each of the medical imaging services provided by the service providers. In such an embodiment, the fees charged by service providers for medical imaging services, included in the service provider information, can not exceed corresponding maximum fees specified by the payor.

The system of the present invention also receives orders, from referrers, for recommended medical imaging services for patients. In accordance with an embodiment of the present invention, the system presents, to a payor, orders for recommended imaging services, to thereby allow the payor (or the payor's designate) to authorize or deny the orders for the recommended imaging service. A payor can also specify that certain types of orders (e.g., orders for X-rays) are pre-authorized.

In response to receiving such an order from a referrer, the system identifies, based on the maintained service provider information, service providers that are capable of performing the recommended medical imaging service. The system also selects at least one service provider from the service providers identified as being capable of performing the recommended medical imaging service. Such selection, in accordance with an embodiment, is based at least in part on the fees charged by the service providers for the recommended imaging service. The selection can also be based at least in part on payor specified rules, details of which are discussed in more detail below.

The selection, in accordance with an embodiment of the present invention, includes ranking the service providers identified as being capable of performing the recommended medical imaging service, based at least in part on the fees charged by the service providers for the recommended imaging service. At least one service provider is then selected based on results of the ranking. In certain embodiments a single service provider is selected by the system. For example, only a highest ranked service provider option may be selected. In alternative embodiments, at least two service provider options are presented to a patient, and the patient is allowed to make the final selection of which service provider option to use. Where the system presents the patient with more than one service provider option, the system may identify the option that is preferred by the payor, and notify the patient of the payor preferred option.

In accordance with an embodiment of the present invention, the system maintains at least one characteristic about each of the medical imaging service providers. Such characteristic(s) can related to, e.g., the location of the service provider, the hours of operation of the service provider, and information about the time it takes for the service provider to complete a medical imaging service. Additionally, the system can maintain one or more payor specified rule relating to one or more characteristic. Such rule(s) can be used to rank the service providers identified as being capable of performing the recommended medical imaging service. In other words, ranking of service providers can be based at least in part on the payor specified rule(s) relating to the at least one characteristic.

In accordance with an embodiment of the present invention, a payor specified rule can be used to filter-out one or more of the service providers identified as being capable of performing the recommended medical imaging service. For example, a rule can specify how far the payor is willing to have a patient travel to get to a service provider to have a recommended imaging service performed. Thus, if a service provider is outside the geographic range specified by the payor, that service provider can be filtered-out. A payor can also specify an exception to a rule, which will prevent a service provider from being filtered-out even though the service provider does not satisfy the rule. For example, a payor may specify that service providers outside of a city's boundaries, or more than 30 miles from a patient, should be filtered-out. A payor may also specify an exception to that rule, which can be, e.g., that a service provider should not be filtered-out if that service provider's fee for the recommended medical imaging service is at least a payor specified amount (e.g., percentage) less than the lowest fee of the service providers within the geographic range of the rule.

In accordance with an embodiment of the present invention, the system maintains information that relates International Classification of Disease (ICD) numbers to Current Procedure Terminology (CPT) codes or other commonly used procedure descriptors. The system allows a referrer to enter an ICD number, and the system identifies based on the maintained information that relates ICD numbers to procedure descriptors (e.g., CPT codes), one or more procedure descriptor (e.g., CPT code) that relates to the entered ICD number. The system can then notify the referrer of the one or more procedure descriptor identified as being related to the entered ICD number, to thereby help the referrer produce an order for an imaging service. It is also within the scope of the present invention to use other disease/condition descriptors besides ICD numbers.

Embodiments of the present invention are also directed to computer implemented systems and methods for reducing costs for obtaining other types of medical services in a supply chain that includes a referrer (e.g., a doctor), a patient, a medical service provider and a payor (e.g., an insurance company) that is responsible for paying at least a part of the fee charged by the service provider for the medical service.

Embodiments of the present invention are also directed to computer implemented systems and methods for reducing costs for obtaining medical goods in a supply chain that includes a referrer (e.g., a doctor), a patient, a medical goods provider and a payor (e.g., an insurance company) that is responsible for paying at least a part of the fee charged by the medical goods provider for the medical good.

This summary is not intended to be a complete description of the various embodiments of the present invention. Further and alternative embodiments, and the features, aspects, and advantages of the present invention will become more apparent from the detailed description set forth below, the drawings and the claims.

### BRIEF DESCRIPTION OF THE DRAWINGS

FIG. 1 is a high level block diagram that is useful for describing an environment in which embodiments of the present invention can be implemented.

FIG. 2 illustrates details of the host system of FIG. 1, according to an embodiment of the present invention.

FIG. 3 illustrates an order sequence 302 associated with a referrer ordering a medical imaging service for a patient, in accordance with an embodiment of the present invention.

FIG. 4 illustrates an exemplary web order form according to an embodiment of the present invention.

FIG. 5 illustrates a scheduling sequence, in accordance with an embodiment of the present invention.

FIG. 6 illustrates an authorization sequence, in accordance with an embodiment of the present invention.

FIG. 7 is a high level flow diagram that is used to describe how systems and methods, according to embodiments the present invention, can be used to reduce the costs for obtaining medical imaging services.

FIG. 8 is a high level flow diagram that is used to describe how systems and methods, according to alternative embodiments of the present invention, can be used to reduce the costs for obtaining medical goods and other types of medical services.

### DETAILED DESCRIPTION

While there have been various attempts to provide online auctions for medical services, some of which are discussed above, such ventures have not been successful in establishing a market. Early ventures offered consumers the ability to shop for low cost providers or procedures (such as plastic surgery) but met with resistance from providers and consumers alike. Objections were based on perceived notions that low cost equates to low quality and that consumers would prefer to seek out physicians based on expertise and experience. These are two important issues that should be addressed and may arise.

First, the notion that consumers are well informed and can choose a physician is generally not the case. The practice of choosing a physician, for the majority of patients, is not based on quality of care. Indeed, the physician's credentials may play only a small role in the selection process. Rather, selection is often dependent on convenience factors such as location and office hours. In the case of medical imaging (e.g., radiological) services, the consumer has little to no information about the imaging provider (e.g., a radiologist) and moreover has almost no role in deciding which imaging provider will be chosen for the imaging services his/her physician has ordered. This degree of separation is critical to the success of an online market for medical services. The consumer, in essence, is marginalized from the decision making process and the ultimate provision of the service. In fact, in imaging there is another set of marginalizing processes that occurs that is beneficial to an online market: the ordering physician has no financial incentive to send a patient to a particular imaging provider and does not control the flow of funds to pay for the service. The second issue raised by the early online medical auction sites, that low cost providers necessarily equate to low quality service, is not a significant barrier in the imaging market. Besides meeting the lowest required standard of acceptable care, there are two primary factors that indicate that the imaging industry will not become a low quality provider of services. These two primary factors are built-in profit and competition.

Payments to imaging providers by third party payors (including Medicare) are above the marginal costs for service. That is, there is a built-in profit margin. This allows providers the option of lowering their costs to the payor, and as seen in competitive markets, this is not an unusual occurrence. There has been continued pressure (lead by Medicare cuts) to reduce the costs of medical imaging services, but this has not lead to a significant consolidation of imaging providers. Rather, in most local markets competition has intensified. To some degree, this has resulted in aggressive pricing practices. However, more often than not, the intensified competition has resulted in imaging providers providing more services and investing in new technology and expertise. The impetus for this continued growth in the face of dropping reimbursement is the shift from “low tech” to “high tech” (and higher reimbursement) imaging. This trend will continue as technology continues to improve the diagnostic tools used in imaging and as physicians continue to rely upon imaging to direct patient care.

Embodiments of the present invention, as will be described below, manage a supply chain that involves four distinct entities that are involved in medical imaging services. These four entities include the referrer, the patient, the provider, and the payor.

The referrer is the entity that specifies that a medical imaging service be provided, and thus, the referrer is the entity initiating the medical imaging service. The referrer is typically a hospital, a physician or other medical practitioner, but is not limited thereto.

The patient is the recipient of the imaging service. In other words, the patient is the entity whose body, or portion thereof, is being imaged. While the patient will typically be a person, it is possible that the patient is an animal.

The provider is the entity that performs the medical imaging service. Accordingly, the term provider can be used to encompass a facility where medical imaging services are performed, the equipment (within a facility) that is used to produce medical images, the personnel that operate the equipment, and the personnel that interpret medical images. In accordance with specific embodiments of the present invention, the term medical imaging service provider will generally refer to the facility where imaging services are performed. Such a facility can be a doctor's office, a hospital, a free-standing imaging center, or a mobile imaging unit (e.g., an ultrasound van).

The payor is the organization that will pay at least a portion of the fees that are charged by the service providers for medical imaging services. For the purposes of this discussion, the payor refers to an entity other than the patient, even though the patient may have a co-payment or a deductible. Additionally, for the purposes of this discussion, the payor is not simply a parent or other guardian of the patient who is willing to pay for the medical imaging services. Rather, the payor refers to an organization that exists at least partially for the purpose of providing medical benefits to patients. Examples of payors include, but are not limited to, health insurance providers, managed care organizations and the government (e.g., through Medicare, Medicaid or veteran's benefits).

Specific embodiments of the present invention relate to methods for essentially performing a reverse auctions for medical imaging services, and systems for performing such methods. As will be described in more detail below, in accordance with specific embodiments the bids in the reverse auctions are automatically generated, based on information previously acquired from service providers. Preferably, embodiments of the present invention reduce the costs to payors (such as insurance companies) and patients for medical imaging services. Preferably, the embodiments of the present invention do not alienate referring physicians and do not adversely affect patient care. Rather, embodiments of the present invention preferably provide for improved patient care and satisfaction.

In accordance with specific embodiments of the present invention, the above is accomplished by using payor specified rules to rank and/or filter-out medical imaging service providers based on key characteristics and/or needs/preferences of the four distinct entities that are involved in medical imaging services. For example, a referrer's needs/preferences can relate to imaging modality and expertise in interpreting images. Patient's needs/preferences can relate to easy access to the provider, a choice of multiple providers and specific conditions for the an imaging exam (e.g., a claustrophobic patient may need/prefer an open bore MRI, as opposed to a closed bore MRI). Characteristics associated with a service provider, include, e.g., the suite of imaging modalities and services offered, specific models of equipment for each modality, professional expertise in obtaining images, professional expertise in interpreting images, hours of operation and location. A payor's needs/preferences include, e.g., lowering the costs of medical imaging exams to reduce spending, and satisfying patients to maintain customers.

While an important feature of embodiments of the present invention is to reduce the cost of imaging for payor's, embodiments preferably also result in the payor selecting a provider that can satisfy the needs of the referrer and patient as well.

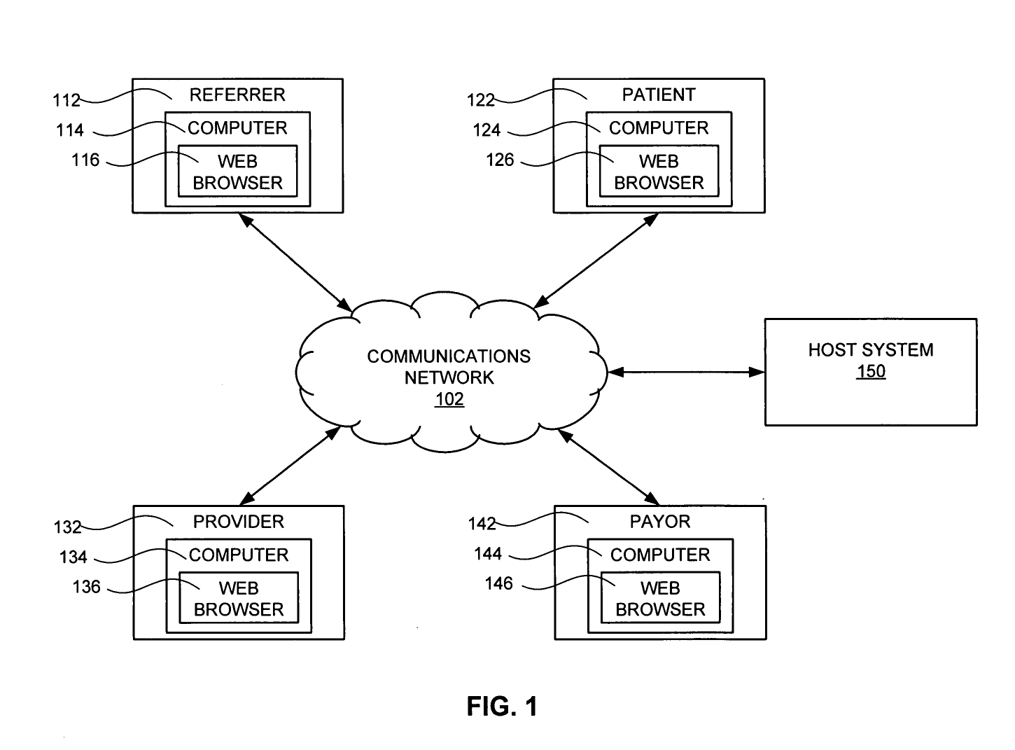

FIG. 1 is a high level block diagram that is useful for describing an environment in which embodiments of the present invention can be implemented. Shown in FIG. 1 is a referrer 112, a patient 122, a medical imaging service provider 132 and a payor 142, each of which is represented by a block. Each of these entities is shown as having access to a respective computer 114, 124, 134 and 144, with each computer preferably having a respective web browser 116, 126, 136 and 146. Through such computers and web browsers, each of the entities 112, 122, 132 and 142 may communicate with one another via the communications system 102, and more importantly, can communicate with the host system 150, which supports the auction capabilities of the present invention.

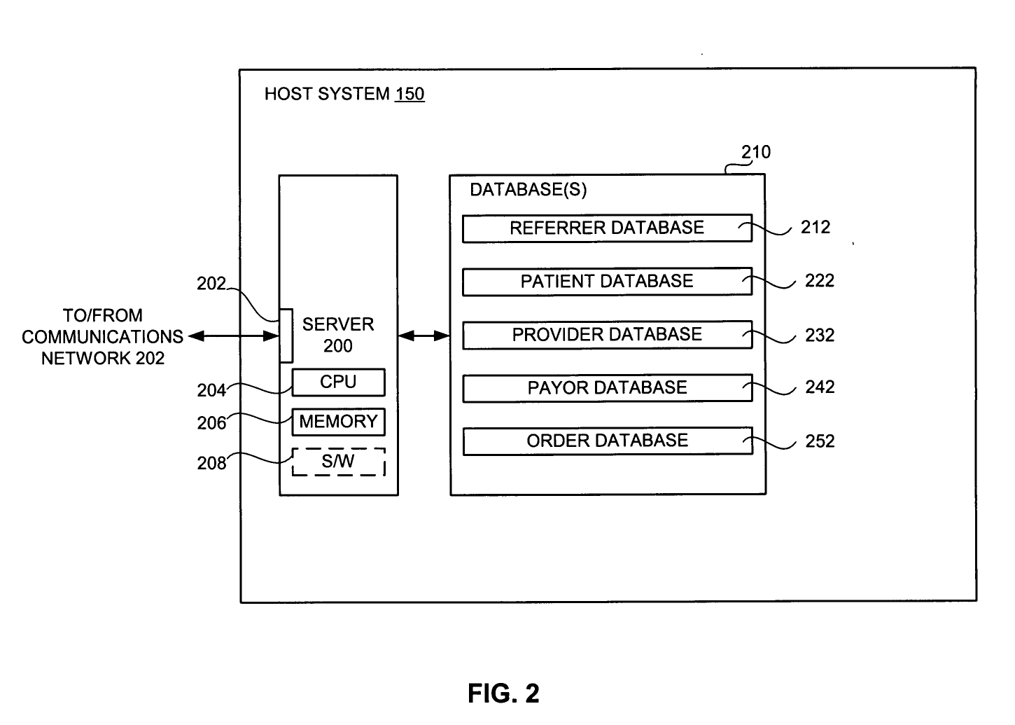

Exemplary details of the host system 150 are shown in FIG. 2. Referring to FIG. 2, the host system 150 can include a server 200 (e.g., a web server) that includes or has access to a communications interface 202, one or more processor 204, memory 206, software 208, a clock (not shown) and one more database 210. The communications interface 202 can allow software and data to be transferred between the host system 150 and external systems. Examples of the communications interface 202 include a modem, a network interface (such as an Ethernet card), a communications port, a PCMCIA slot and card, etc. Software and data transferred via the communications interface 202 are in the form of signals which may be electronic, electromagnetic, optical or other signals capable of being received by communications interface. These signals are provided to communications interface 202 via a communications path, which can be implemented using wire or cable, fiber optics, a phone line, a cellular phone link, an RF link and other communications channels.

In this document, the terms “machine readable medium”, “computer program medium” and “computer usable medium” are used to generally refer to media such as removable storage drive, a hard disk installed in hard disk drive, and signals. These computer program products are means for providing software 208 to the host system 150. Computer programs (also called computer control logic) are stored in memory 206 or removable storage units (not shown). Computer programs may also be received via the communications interface 202. Such computer programs, when executed, enable the host system 150 to implement specific features of the present invention as discussed herein. In particular, the computer programs, when executed, enable the one or more processor 204 to implement the features of the present invention. Where the invention is implemented using software, the software 208 may be stored in a computer program product and loaded into the host system 150 using a removable storage drive, a hard drive or the communications interface.

The database 210 can be made up of separate databases, or separate portions of the database 210. Exemplary portions of the database, or separate databases, include a referrer database 212 (or database portion), a patient database 222 (or database portion), a provider database 232 (or database portion), a payor database 242 (or database portion) and an order database 252 (or database portion). The referrer database 212 can store information (e.g., profiles) about each referrer, including, but not limited to, their name, address and contact information. The patient database 222 can store demographic information about each patient, including but not limited to, their name, address and contact information. The patient database 222 can also store insurance information, information about allergies, biometric information (e.g., date of birth, weight, height), and medical history information (e.g., by ICD code). Additionally, the patient database 222 can also store more subjective information about each patient, such as doctor preferences, whether the patient participates in athletics, whether a patient is claustrophobic, etc.

The provider database 232 can store information about each medical imaging provider, including but not limited to, their name, location (e.g., address), hours of operation, contact information and information about the insurance they accept. The provider database 232 can also include, for each service provider, a list of medical imaging services (e.g., imaging modalities) that are provided by the service provider and a corresponding fee charged for each of the medical imaging services. The fee charged for each medical imaging service can be a global fee, or can be split into a professional fee portion and a technical fee portion (with the sum of the professional and technical fees being a global fee). Being able to specify split fees is useful where a payor requires such split fee type information from service providers.

In accordance with an embodiment, each list of medical imaging services can include, e.g., a list of Current Procedure Terminology (CPT) codes or other procedure descriptors. Preferably a list of procedure descriptors (e.g., CPT codes) is maintained and presented to a service provider, thereby enabling the service provider to specify (e.g., by checking a box, or the like) the procedure descriptors corresponding to the imaging services that the provider can perform.

There is also the option that a service provider can specify their fee for an imaging service (or more specifically, for a CPT code or other procedure descriptor associated with the imaging service) by specifying a percentage of a fee specified in the Medicare fee schedule, which is updated once a year.

Additionally, the provider database 232 can include information about the expertise of each provider and information about the imaging equipment (e.g., year, model, brand) of each provider. Where a provider has one or more mobile imaging unit (e.g., an ultrasound van or MRI/CT/PET truck), such providers can assign daily locations to each unit, and each unit can be treated as a fixed unit (for each day), thereby enabling such service providers to participate in the auction of the present invention.

The payor database 242 can store information about each payor, including but not limited to their name, address and contact information. The payor database 242 can also include, for each payor, a list of service providers that are within the payor's network. Additionally, the payor database 242 can include, for each payor, a list of medical imaging services that are at least partially covered by the payor and a maximum fee that the service providers can charge for each medical imaging service. In accordance with an embodiment, each list of medical imaging services can be, e.g., a list of Current Procedure Terminology (CPT) codes. In accordance with an embodiment, the maximum fee information can be a payor specified dollar amount. In an alternative embodiment, the maximum fee information can be payor specified percentage amounts of the Medicare fee schedule. For example, a payor may specify that the maximum fee for an MRI is equal to 80% of the Medicare fee schedule amount; and that the maximum fee for a CT is equal to 105% of the Medicare fee schedule, etc. The payor can change such maximum fee information through the payor's web account.

The payor database 242 can also include, for each payor, rules that are specified by the payor for use in filtering-out and/or ranking service providers. As will be describe in more detail below, the payor database 242 can also include exceptions to such rules, which are also specified by the payor, where the exceptions will prevent a service provider from being filtered-out. Additionally, the payor database 242 can specify which medical imaging services are pre-authorized, and which services require authorization on a case by case basis. This can be on a service provider basis, or on a global basis. The payor can change such rules through the payor's web account.

Referring back to FIG. 1, a payor 142, through use of a web browser 146, will be able to change their information stored in the payor database 242. For example, a payor can change the maximum fees that service providers within the payor's network can charge for medical imaging services. The payor can also change their rules, as well as their exceptions to their rules. This will be explained in more detail below.

Returning to FIG. 2, the order database 252 can store orders for medical imaging services for patients. Such orders, as will be described below, are produced by a referrer. The order database 252 can also specify whether each order has been authorized by the payor or is pending authorization. An order may be authorized because the medical imaging service included in the order has been pre-authorized by the payor, or because the payor has reviewed the order and authorized it. The payor can access the order database to review orders and either authorize them or deny them. When an order has been authorized or denied by the payor, the order database 252 is updated to include such information. Such information is also used to update appropriate web accounts, as will be described below. In accordance with specific embodiments of the present invention, the payor can designate that another entity (referred to hereafter as a “payor's designate” or a “designate of the payor”) be responsible for the authorization of medical imaging services. For example, an insurance company payor can designate that a radiology benefits management (RBM) company have the responsibility for authorizing or denying ordered medical imaging services. In such embodiments, the payor's designate (e.g., an RBM) would have access to the order database to review orders and either authorize them or deny them. A payor can provide it's designate with as much responsibility as the payor desires. Thus, in accordance with specific embodiments of the present invention, any payor specified rule or exception discussed below may actually be specified by a designate, assuming the payor gave the designate authorization to control such rules or exceptions.

The referrers, patients, payors and medical imaging service providers can each have their own web account that enables these entities to view and update certain information using a web browser. Each web account can have a password that is set up by the entity corresponding to the account, thereby limiting access to the web accounts. Such web accounts are likely hosted by the host system 150. The web accounts can be stored within the various databases or have access to the various databases (or database portions) shown in FIG. 2.

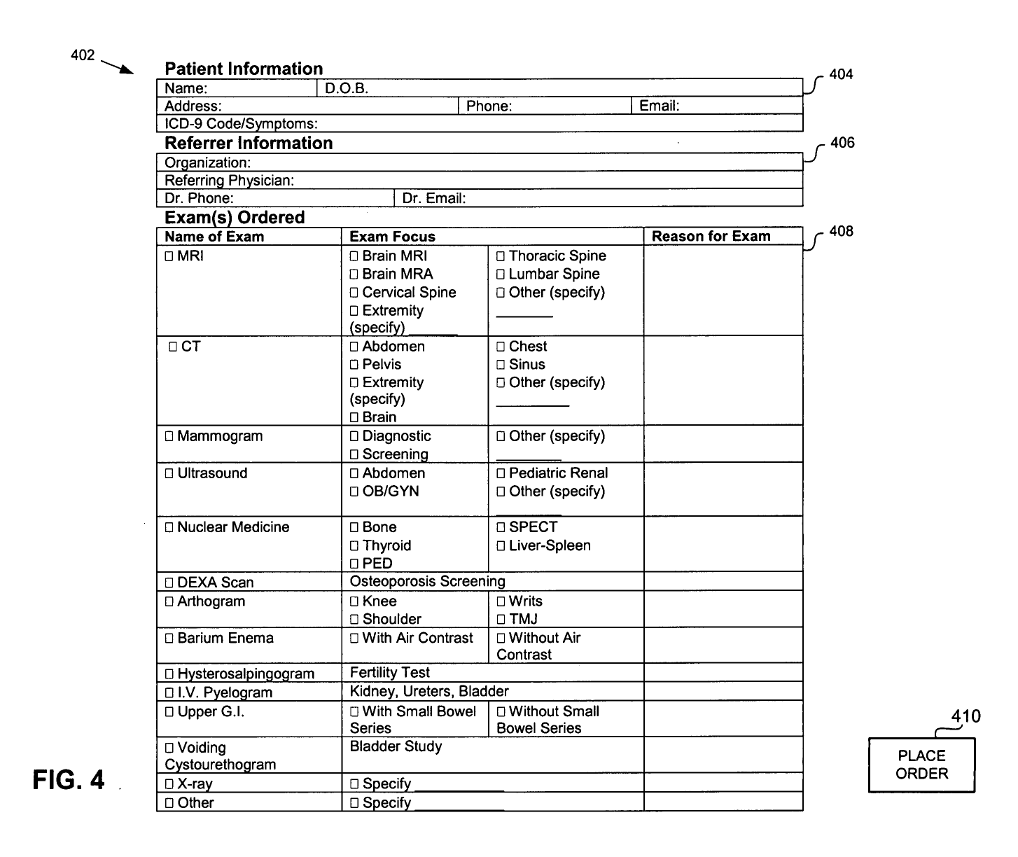

The web account for a referrer can enable the referrer to place orders for medical imaging services (e.g., imaging exams). In one embodiment, a referrer can access a web order form to order a medical imaging service. An exemplary web order form is shown in FIG. 4. The referrer can also specify a time to complete, which is the number of days (or other unit of time) within which an ordered medical imaging service must be performed. The web account for a referrer may also provide the referrer with metrics that were generated by the host 150 for that referrer, including but not limited to: average volume and variance volume per service (e.g., by CPT code); average volume and variance volume per diagnosis (e.g., by ICD code); exam order metrics for the referrer; and demographic spread of payors, providers and patients. The web account for a referrer may also allow the referrer to view the status of orders, including, e.g., whether the orders have been authorized or denied, and whether the orders have been completed (i.e., whether an order medical imaging service has been performed). In a specific embodiment, the web account for a referrer provides the referrer with results of a medical imaging service, e.g., links to images and/or interpretations of images.

The web account for a patient enables the patient to review medical imaging services that have been ordered for the patient, as well as whether the services have been authorized or denied by the payor. Scheduling information can also be provided in a patient's web account. The web account for a patient can also allow the patient to update information stored in the patient database (or database portion) 222, such as demographic information, insurance information, information about allergies, biometric information, medical history information, doctor preferences, whether the patient participates in athletics, whether a patient is claustrophobic, etc. In alternative embodiments, some of the above information can only (or also) be updated by one of the other entities.

The web account for a service provider informs the service provider of the medical imaging services that the provider has been selected to perform, whether such services have been authorized by payor, information about the patient's that correspond to the imaging services to be performed, and information about whether and when a medical imaging service has been scheduled. The web account for a service provider can also allow the service provider to change or add characteristics about the service provider (e.g., if the service provider changes their hours of operation, equipment, personnel, fees etc.) The web account for a service provider may also provide the service provider with metrics that were generated by the host 150, including but not limited to: average revenue, volume and variance in revenue and volume per service (e.g., by CPT code); average revenue, volume and variance in revenue and volume per diagnosis (e.g., by ICD code); exam order metrics per referrer or specialty; and demographic spread of payors, providers and patients.

The web account for a payor can include information about all the medical imaging providers, patients and/or referrers within the payor's network. The web account for the payor can also allow the payor to view orders and authorize or deny each order. The web account for a payor can also allow the payor to specify the maximum fee that service provider's within the payor's network can charge for each of the medical imaging services at least partially covered by the payor. The web account for the payor can also allow the payor to specify rules that are used to select one or more service provider to fulfill an order for a medical imaging service, as well as to identify a preferred service provider. The web account for a payor can also allow the payor to specify exceptions to rules, as will be described in more detail below. Additionally, the web account for a payor can allow the payor to indicate weighting factors that can be used in ranking service providers, where such ranking is used in the selection process. The web account for a payor may also provide the payor with metrics that were generated by the host 150, including but not limited to: average savings, cost, volume and variance in savings and cost volume per service (e.g., by CPT code); average savings, cost, volume and variance in savings and cost volume per diagnosis (e.g., by ICD code); exam order metrics per referrer or specialty; and demographic spread of payors, providers and patients. Where the payor has designated that another entity (e.g., an RBM) handle the authorization of ordered medical imaging services, the payor's designate can have access to their own web account that enables the designate to access the order database to authorize or deny orders. Alternatively, the payor's designate may have access to certain portions of the payor's web account.

In specific embodiments, service providers and payors can bundle groups of procedure descriptors (e.g., CPT codes) together for a single price. For example, a CT scan of the chest, abdomen and pelvis can be assigned one price instead of three for its three CPT codes. This grouping may be referred to as “contiguous body part imaging”, which refers to the situation where multiple body parts are imaged in one single setting. Service providers can also have a non contiguous bundling option. For example, a service provider can bundle an ultrasound of the kidneys and an ultrasound of the pelvis for general abdominal pain. For another example, a service provider can bundle an MRI of the cervical spine and an MRI of a shoulder for pain relating to an arm. More generally, symptoms can be used as a focal point for bundling exams together. The providers and payors can establish these sorts of bundles in their fee lists.

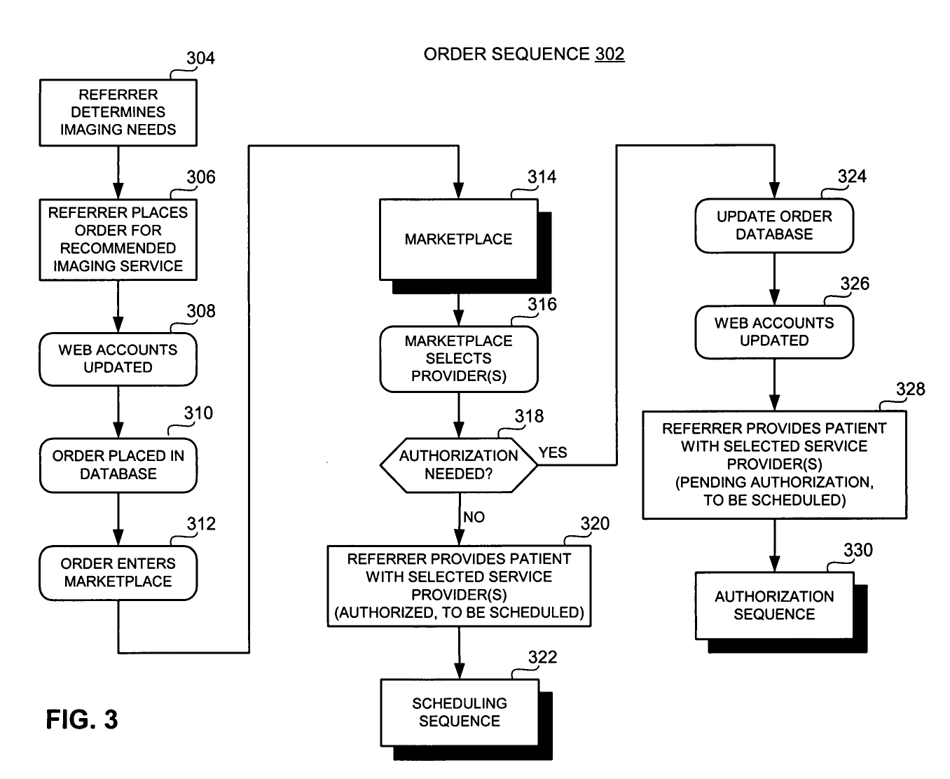

Referring now to FIG. 3, shown therein is order sequence 302 associated with a referrer ordering a medical imaging service for a patient. At block 304, a referrer determines the medical imaging needs for the patient, e.g., by examining the patient and reviewing the medical history of the patient. Next, at a block 306, the referrer logs onto a web account supported by the host 150, to thereby place an order for a recommended medical imaging service for the patient. An exemplary order webpage or webform, which can be used to assist the referrer in placing the order, will be described below with reference to FIG. 4. At that point, web accounts (e.g., of the referrer, patient and payor) are updated to include information about the order, as specified at block 308. As mentioned above, such web accounts can be stored, e.g., in the databases (or database portions) described with reference to FIG. 2. This can include, as specified at block 310, including the order in the order database 152. As shown at block 312, the order then enters the marketplace of the present invention, which is represented by block 314. The marketplace of the present invention selects one or more medical imaging service provider, as indicated by block 316. Embodiments of the present invention that are used to perform such selection will be described in detail below.

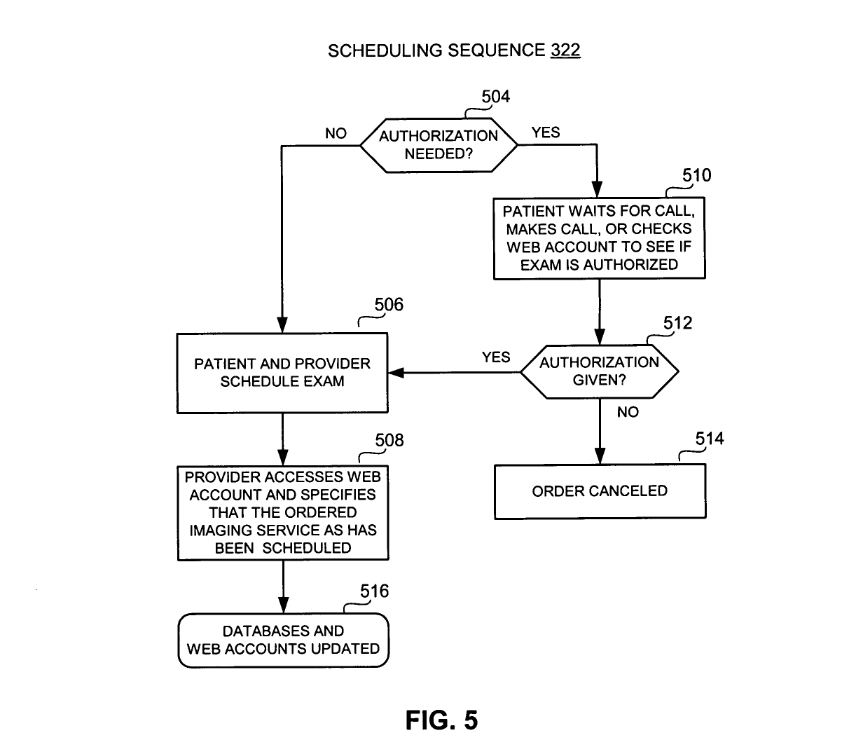

Still referring to FIG. 3, at block 318 there is a determination of whether authorization from the payor is needed for the ordered medical imaging service. If authorization for the ordered medical imaging service is not needed from the payor (e.g., because the payor has pre-authorized the ordered medical imaging service), then the referrer provides the patient with information about the selected one or more medical imaging service provider that may perform the ordered medical imaging service, as indicated at block 320. This can include, e.g., providing the patient with a printout of such information and/or emailing such information to the patient. A scheduling sequence 322 then occurs, which is described in more detail below with reference to FIG. 5.

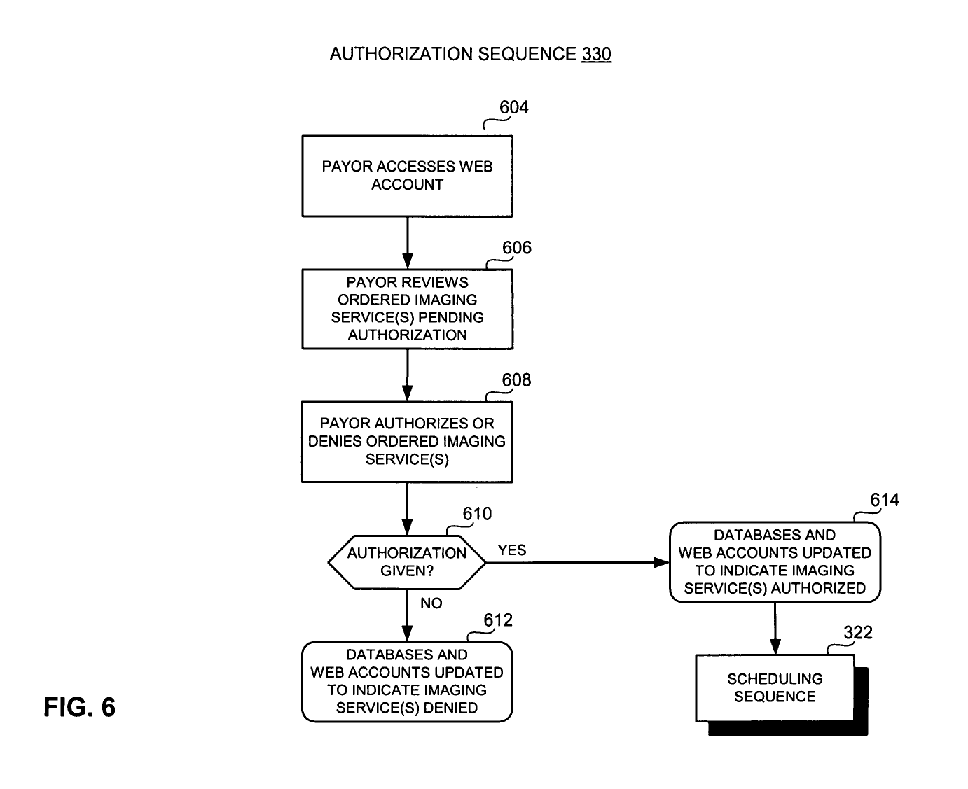

Returning to block 318, if authorization is needed, then the order database (or database portion) 252 is updated to specify that the order is pending authorization. At this point web accounts of the referrer, patient, selected service provider(s), and payor can be updated to specify that the order is pending authorization from the payor, as indicated at block 326. As indicated at block 328, the referrer can then provide the patient with information about the selected one or more medical imaging service provider that may perform the ordered medical imaging service, pending authorization. This can include, e.g., providing the patient with a printout of such information and/or emailing such information to the patient. An authorization sequence 330 then occurs, which is described in more detail below with reference to FIG. 6.

As mentioned above, FIG. 4 illustrates an exemplary web order form 402 that can be presented to a referrer when the referrer logs onto its web account, e.g., using a web browser. Such a web order form 402 can include fields 404 where the referrer can enter patient information, and fields 406 where the referrer can enter referrer information. The order form 402 can also include, within section 408, boxes that the referrer can check to select an exam name and an exam focus. There is also room for the referrer to specify other types of exam focuses not specifically listed, as well as room for the referrer to type in reasons for exams. The form 402 also includes a button or other interface 410 that can be pressed or otherwise selected by the referrer (e.g., using a mouse or other interface device) to place an order. Once the order is placed, the form 402 and/or information therein can be stored in the order data base 252, where it can be accessed by the referrer, as well as by the payor to assist the payor in determining whether to authorize or deny the order.

FIG. 5 illustrates the scheduling sequence referred to at block 322 in FIG. 3. Referring to FIG. 5, at block 504 there is a determination of whether authorization is needed, which it may not be if the payor has indicated that a specific ordered medical imaging service is pre-authorized, which may be the case for relatively inexpensive imaging services such as X-rays. If authorization is not needed, then the patient and service provider schedule the ordered medical imaging service, e.g., over the phone, by email, or the like, as indicated at block 506. The provider then accesses its web account to specify that the ordered imaging service has been schedule, as indicated at block 508. The appropriate database(s) and web accounts are then updated, as specified at block 516. Returning to block 504, if authorization has not yet been provided, then the patient should wait until authorization is provided to schedule the imaging service. If the payor denies the authorization, then the order is canceled, as indicated at block 514.

FIG. 6 illustrates the authorization sequence referred to at block 330 in FIG. 3. Referring to FIG. 6, at block 604 the payor accesses the payor web account, and at block 606 the payor reviews the ordered medical imaging services that are pending authorization. The payor then decides, at block 608, whether to authorize or deny ordered medical imaging services. At block 610 there is a determination of whether authorization has been given. If authorization is denied for a medical imaging service, then appropriate databases and web accounts are updated to indicate such, at block 612. If authorization is granted, then appropriate databases and web accounts are updated to indicate such, as indicated at block 614, and then the scheduling sequence 322 occurs, which was described above with reference to FIG. 5.

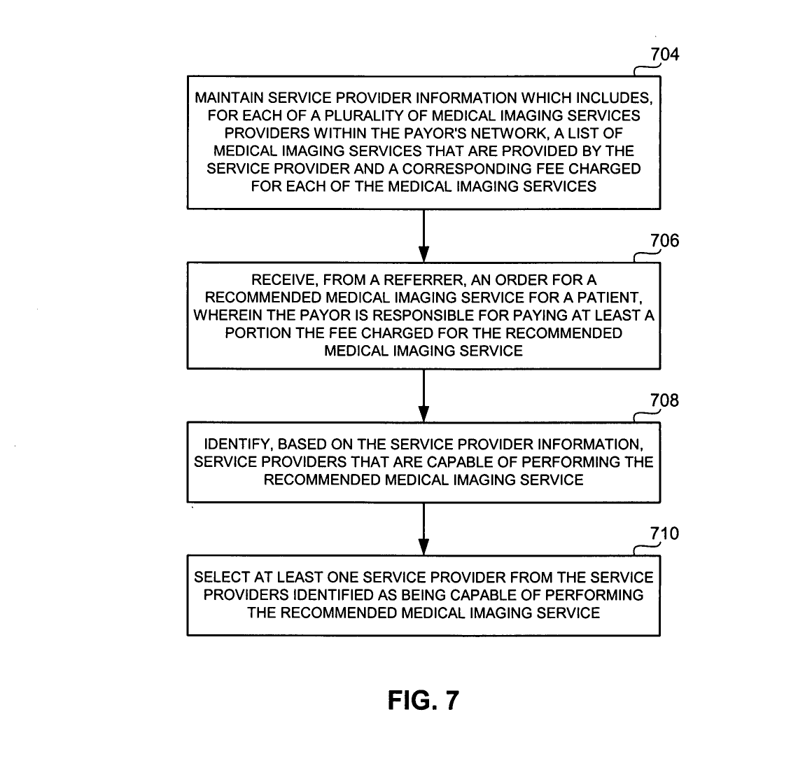

FIG. 7 is a high level flow diagram that is useful for describing how one or more medical imaging service provider can be selected to perform a medical imaging service, in accordance with an embodiment of the present invention. The steps of FIG. 7 are explained from the perspective of the host 150, which will host the hardware and software that can be used to implement the steps of FIG. 7.

Referring to FIG. 7, step 704 includes maintaining service provider information which includes, for each of a plurality of medical imaging services providers within the payor's network, a list of medical imaging services that are provided by the service provider and a corresponding fee charged for each of the medical imaging services. Such information is likely maintained by the host 150, within the database(s) 210. In addition to maintaining a list of imaging services (e.g., modalities) and the fee for each service, further types of service provider information, referred to as characteristics or characteristic information, can also be stored for each service provider. Such characteristics can include, but are not limited to, the location and hours of operation of the service provider, as well as information (e.g., historical information) about the time it takes for the medical imaging service provider to schedule a medical imaging service and/or complete a medical imaging service. Other characteristic information that can be maintained for each service provider includes information about the provider's imaging and post processing equipment, such as, but not limited to, model numbers, manufacturer, age of equipment, three-dimensional capabilities, and the like. Further characteristics that can be maintained for each service provider include the names of physicians and technologists, expertise of the physicians and technologists, certifications of physicians and technologists and the like. Service provider characteristics can also relate to whether the provider uses image transport services, e.g., courier services, and/or whether they provide on-line access to images e.g., using a Picture Archive Communication (PAC) system. Service provider characteristics can also relate to how medical imaging service providers provide access to image reports, e.g., via mail, facsimile and/or online through a Radiology Information System (RIS) or a PAC system. Another characteristic can relate to whether the medical imaging service providers provide patient transportation (i.e., pick-up and drop-off services), which is very usefull when the patient is elderly or disabled. Another characteristic can relate to whether the medical imaging service provider reserves emergency slots, which can be important if a referrer needs a medical imaging service performed immediately (e.g., if a patient was just in a serious accident, or the like) Information about patient satisfaction and referrer (e.g., physician) satisfaction are examples of still other characteristics that can be maintained for each service provider. Certain one's of the above mentioned characteristics, such as fees charged and hours of operation, can be changed by a service provider, e.g., through use of the service provider's web account. Other characteristics, such as patient satisfaction, are based on feedback from patients.

Still referring to FIG. 7, step 706 includes receiving, from a referrer, an order for a recommended medical imaging service for a patient, wherein the payor is responsible for paying at least a portion of the fee charged for the recommended medical imaging service. As was described in detail above, the referrer can place such an order by accessing its web account and, e.g., filling out a web order form, an example of which was described above with reference to FIG. 4. Once the order is placed, the host 150 receives the order.

In accordance with an embodiment of the present invention, the host system 150 maintains information that relates International Classification of Disease (ICD) numbers to Current Procedure Terminology (CPT) codes or other procedure descriptors. The host system 150 can also provide a user interface (e.g., a graphical user interface) to referrers, which allows a referrer to enter an ICD number or other disease/condition descriptor using their web account. The host system 150 can then identify, based on the information that relates disease/condition descriptors (e.g., ICD numbers) to procedure descriptors, one or more procedure descriptor (e.g., CPT code) that relates to the entered disease/condition descriptor. The host system 150 can also notify the referrer through their web account of the one or more procedure descriptor (e.g., CPT code) identified as being related to the entered ICD number (or other disease/condition descriptor), to thereby help the referrer produce their order. In this manner, the host system can steer the referrers to the most appropriate imaging services for a diagnosis. Some procedures, such as a pituitary MRI and a brain MRI share the same CPT code. Accordingly, in certain embodiments, the procedure descriptor used enables such procedures to be distinguishable, even though they share the came CPT code.

Step 708 includes identifying, based on the maintained service provider information, service providers that are capable of performing the recommended medical imaging service. For example, if the recommended medical imaging service is a lumbar spine MRI, the service provider information is queried to identify those service providers within the payor's network that are capable of providing that service. In this manner, the service providers that are not capable of performing the recommended imaging service are in essence filtered-out, thereby narrowing down the pool of possible service providers. In other words, step 708 can be thought of as specifying which service providers can bid to perform the recommended medical imaging service for a patient.

Step 710 includes selecting at least one service provider from the service providers identified as being capable of performing the recommended medical imaging service. In accordance with an embodiment of the present invention, the selecting in step 710 is based at least in part on the fees charged by the service providers for the recommended imaging service. This can include, for example, ranking the service providers identified as being capable of performing the recommended medical imaging service, based at least in part on the fees charged by the service providers for the recommended imaging service, and then selecting the at least one service provider based on results of the ranking.

If cost is the only factor taken into account in step 710, then the service provider that charges the lowest fee for the recommended imaging service can be selected. In specific embodiments, a payor can provide the patient with a payor specified number of service provider options. For example, the payor can specify that it will provide three difference service provider options to the patient, and thereafter let the patient make the final selection of which service provider to use. Where the payor gives the patient multiple options, the patient can be notified of the payor's preferred option, e.g., through use of highlighting, asterisking, or the like. At the option of the payor, the payor may also inform the patient that the payor will waive or lower a deductible or co-payment, if the patient selects the payor's preferred option. Alternatively, where a patient is responsible for paying a percentage (e.g., 20%) of a fee, the payor may inform the patient that the payor will lower the percentage if the patient selects the payor's preferred option. Alternatively, where a payor's preferred option is the least expensive option, a patient responsible for paying a percentage (e.g., 20%) of a fee may simply be inclined to select the payor's preferred option because it will also be the least expensive option for the patient.

When the patient is given multiple options, additional information can be provided to the patient (e.g., via the patient's web account, or via a printout provided by the referrer to the patient) to assist the patient in making the final selection. For example, the location and hours of operation of each service provider option can be provided to the patient. The travel distance from the patient to each service provider option can also be determined and provided to the patient. The patient can also be provided with the option of specifying the specific address (e.g., home or work address) from which the travel distance should be calculated. Patient satisfaction information for each service provider option can also be provided to the patient.

In specific embodiments, other characteristics of the service providers, in addition to cost, are taken into account during the selecting performed at step 710. As explained above, other characteristics can include, but are not limited to, location of the service providers (e.g., for using in determining travel distance), hours of operation of the service providers, patient satisfaction for the service providers, and the like. In specific embodiments, the payor can assign weights to such characteristics, whereby the weights are used to rank the service providers that are capable of performing a recommended medical imaging service. Then, the highest ranked service provider can be selected at step 710. Alternatively, as described above, a payor specified number of highest ranked service providers can be selected for presentation to the patient, thereby allowing the patient to make the final selection. Also, as described above, a payor's preferred service provider option can also be identified and specified to the patient.

Specifying how many options will be presented to a patient is an example of a payor specified a rule. The payor's assigning of weights to various characteristics is another example of a rule that can be specified by the payor. These and other rules can be specified by the payor, and changed by the payor, through uses of the payor's web account. Other rules can be more definitive, such as, a rule that filters-out all service providers outside of a specific geographic region or proximity, or filters-out all service providers where the travel distance for the patient is beyond a payor specified threshold. As is apparent, the locations of the service providers and the patient are used when applying such rules. Another rule can filter-out all service providers having an average time to completion that exceeds a payor specified threshold. As will be described in more detail below, the payor can also specify one or more exception to a rule.

An exception to a rule prevents a service provider (that is capable of performing a recommended medical imaging service) from being filtered-out, even if the service provider does not otherwise satisfy the rule. For example, a payor may specify that service providers outside of a city's boundaries, or more than 30 miles from a patient, should be filtered-out. A payor may also specify an exception to that rule, which can be, e.g., that a service provider should not be filtered-out if that service provider's fee for the recommended medical imaging service is at least a payor specified amount (e.g., percentage) less than the lowest fee (for that service) of the service providers within the geographic range of the rule. For example, assume that the lowest fee charged by a service provider that satisfies the rule is $1000, but that there is another service provider 35 miles from the patient having a fee of $600 for the recommended imaging service. In this instance, an exception to the rule can enable the payor to select the service provider having the $600 fee, because the payor may feel that its worth having the patient travel a little further (possible as little as 5 miles) to save $400. It is also within the scope of the present invention that the payor can specify granularities for an exception. For example, it may be that the further a service provider is from the patient, the lower the fee of that service provider must be to prevent that service provider from being filtered-out. There are numerous ways such rules can be implemented, all of which are within the scope of the present invention. Payor specified exceptions can also be non-monetary. For example, the payor may specify that certain rules be relaxed (e.g., filters widened) for service provider's having high levels of patient satisfaction.

Other payor specified rules and exceptions can relate to referrer preferences. For example, a referrer may order a closed bore MRI, as opposed to an open bore MRI, because the closed bore MRI provides better resolution. Depending upon the reason for the exam, the payor may specify that a service provider capable of providing an open bore MRI service should be selected to fulfill the ordered service, if the fee charged by that service provider for the open bore MRI is at least a payor specified amount less than the lowest fee charged for a closed bore MRI. However, if the order for the closed bore MRI specifies that the closed bore MRI is absolutely necessary, e.g., because an open bore MRI would not have sufficient resolution to detect what is being imaged, then all service providers not capable of providing a closed bore MRI should be filtered-out. In another example, a payor specified rule may specify that certain preferences by the referrer must be accepted. For example, if a referrer orders an open bore MRI because the patient is claustrophobic, assuming such reason for the open bore MRI is noted in the order, the payor will filter-out all service providers not capable of providing an open bore MRI, even if the cost of a closed bore MRI is significantly less than the least expensive open bore MRI.

For another example, a referrer may specify that a medical imaging service must be completed within a certain time frame (e.g., within 2 days), because the patient is scheduled for surgery on a specific day (e.g., in 3 days) at a certain time. A payor specified rule would preferably specify that completion of the service within the time frame is a necessary requirement. More generally, embodiments of the present invention allow referrers to specify preferences, and payors to specify rules that indicate how referrer preferences are treated or weighted (e.g., not at all, lightly, heavily, absolutely). Such rules can be common for all referrers, or on a referrer by referrer basis, depending on how the payor wants to define the rules. In accordance with an embodiment, a referrer can specify their preferences using the referrer's web account, and a payor can specify how they treat referrer preferences using the payor's web account.

Additionally, embodiments of the present invention allow patients to specify their preferences, such as whether they prefer open bore machines because they are claustrophobic, whether they need transportation to and from an imaging service provider (e.g., because they are elderly or disabled), when they want imaging services performed, what's the maximum distance they want to travel, whether they prefer certain providers, and the like. Further, embodiments of the present invention allow payors to specify rules that indicate how patient preferences are treated or weighted (e.g., not at all, lightly, heavily, absolutely). In accordance with an embodiment, a patient can specify their preferences using the pateint's web account, and a payor can specify how they treat patient preferences using the payor's web account. Allowing payors to take into account referrer and patient preferences will increase referrer and patient satisfaction and minimize referrer and patient backlash.

What occurs at step 710 can be thought as selecting service providers based on automatically generated bids (from service providers) for the recommended medical imaging service, where the automatic bids are produced based on maintained information previously acquired about (and typically from) medical imaging service providers. In other words, service providers use their web accounts to enter information, such as lists of the medical imaging services they provide and the fees charged for the services, equipment information, hours of operation information, location information, etc. Other characteristic information, such a patient satisfaction and average time of completion can also be acquired about each service provider. Then, when a referrer orders a medical imaging service for a patient, bids from service providers (that are capable of performing the service) are essentially automatically generated based on the maintained information about the medical imaging service providers, and one or more service provider is selected based on such automatic bids, using payor specified rules.

A benefit of having bids automatically generated is that a referrer, within a very short time (e.g., seconds or minutes) of ordering an imaging service, can provide a patient with the name of one or more service providers (possibly pending authorization by a payor) that the patient can visit to have the ordered imaging service performed. This is advantageous over other auction methods, where it may take a relatively long time (e.g., days or weeks) before all bids are collected. Nevertheless, in an alternative embodiment of the present invention, requests for bids can be provided to service providers that are capable of performing the ordered imaging service, and the service providers can produce bids on an order-by-order basis. That is, in this alternative embodiment, bids are not automatically generated based on information maintained by the host.

Returning to the discussion of the primary embodiments of the present invention (where bids are automatically generated based on information maintained by the host 150), payor specified rules can be turned on, turned off, or changed by the payor, through use of the payor's web account. Additionally, payor specified rules can be prioritized by the payor, through use of the payor's web account. Further, the number of service options that will be provided to a patient can be changed by the payor, using the payor's web account.

For example, assume that a payor specifies that a patient will be given two service provider options from which the patient can make the final selection. Also assume that the payor has defined three rules for use in selecting which two service providers options to present to the patient. The three rules can relate to, e.g., location of the service provider, hours of operation of the service provider, and patient satisfaction. The location rule may specify that a service provider more than 20 miles from the patient should be filtered out. The hours of operation rule may specify that a service provider that is not open on a weekend should be filtered out. The patient satisfaction rule may specify that higher satisfaction ratings are preferred. In accordance with an embodiment, the payor can specify the order in which the rules will be serially applied, in the selection process. For example, assume that there are ten imaging service providers capable of performing the recommended imaging service, and that the ten should be narrowed down to two service provider options. The payor may specify that the location rule be applied first, followed by the hours of operation rule, and then followed by the patient satisfaction rule. Exceptions, examples of which were discussed above, can also be associated with each rule. By appliing the rules in a payor specified order, the starting pool of ten providers is narrowed down. If more than two options remain after the rules are applied, then the two most inexpensive remaining options may be presented to the patient.

In accordance with an embodiment, a payor or designate can specify that rules stop being applied once the pool of service providers capable of performing a recommended medical imaging service is narrowed down to the number of options specified by the payor. For example, it may be that after the first two rules are applied only two service provider options remain. At that point, the third rule need not be applied.

In accordance with an alternative embodiment, rather than applying rules serially, rules can be applied in parallel. In one embodiment, this can be combined with allowing a payor to specify weights for different rules.

In accordance with an embodiment, a payor or designate can opt to allow the system to automatically change rules and/or change the order of rules, to assure that the payor specified number of service provider options can be selected. For example, it may be that if payor specified rules were applied in the order specified by the payor that only one service provider option satisfied all of the rules, in the order specified, but that the payor wanted to present a patient with two options. If authorized to do so by the payor, the host system can automatically change certain rules and/or the order of rules in an attempt to present the patient with two options. Such automation can be relatively simple (e.g., the system can change the order of the first two rules), or as complicated as desired.

Rules that are used to filter-out service providers can be referred to as “filter rules” or simply “filters”, because they f unction much like a filter in that service providers within scope of the filter pass through the filter, while service providers that are outside the scope of the filter are filtered out. Changing such rules can also be referred to as “modulating” the rules or modulating the filters. Similarly, where payor specified rules take into account referrer and/or patient preferences, it can be said that the rules or filters are modulated based on such preferences. Because payors can specify how to treat referrer and/or patient preferences, the payors can control the extent of such modulating.

In specific embodiments, a payor can specify a rule that alters (e.g., increases) the price the payor pays a service provider for an ordered medical imaging service. Such rules can be to reward service providers. For example, if a service provider satisfies all the payor's rules related to price and other characteristics, the payor can have the option of adding a small percentage to the fee the service provider charges, thereby overcompensating the service provider as a reward. This can be referred to as pay for performance (P4P) reward. An alternative reward may be to favorably alter an exception to a rule for only specific service providers. For example, where a service provider consistently has high patient satisfaction scores (or a combination of certain characteristics, such as low turn around time and high patient satisfaction scores), the payor can alter a distance rule/filter by in essence making the provider “5 miles closer,” or by reducing the percentage lower fee exception to the rule/filter so that rather than needing to have a 20% lower fee if outside the distance rule/filter, this service provider needs to have only a 15% lower fee.

After an order for a recommended imaging service is authorized, a service provider can fulfill (i.e., complete) the ordered service, cancel the service or submit a change order. Also, the referrer can cancel or submit a change order, after an order has been made, but not yet completed.

Assuming a medical imaging service is completed (i.e., performed by the medical imaging service provider), the service provider accesses its web account and specifies that the medical imaging service has been completed, which will cause the appropriate databases and web accounts to be updated accordingly. Results of the medical imaging service can then be provided to the referrer and/or patient through their respective web account, by email, by regular mail, by phone, or any other appropriate manner.

A service provider or referrer, as mentioned above, may also cancel an order, e.g., if the service provider determines that the ordered service is inappropriate, dangerous to the patient, or the like. To cancel an order, the service provider or referrer logs onto their respective web account and specifies that the order is canceled, which will cause the appropriate databases and web accounts to be updated accordingly.

Instead of canceling an order, a service provider or a referrer may submit a change order, if one of them determines that a different medical imaging service is more appropriate than the service originally ordered. To change an order, the service provider or referrer accesses their respective web account, and submits a change order request, which will cause the appropriate databases and web accounts to be updated accordingly. As with an original order, the changed order can be pre-authorized, or may require specific authorization by the payor. If the changed order requires authorization by the payor, then the payor when reviewing its web account will either authorize or deny the ordered medical imaging service, in a similar manner as was described above with reference to FIG. 6.

Post market information can be maintained and continually updated by the host system 150. For example, as mentioned above, metrics that are generated by the host 150 for the referrer can include, but are not limited to: average volume and variance volume per service (e.g., by CPT code); average volume and variance volume per diagnosis (e.g., by ICD code); exam order metrics for the referrer; and demographic spread of payors, providers and patients. Preferably, such information is made available to the referrer through the referrer's web account. Similarly, as mentioned above, metrics that are generated by the host 150 for a service provider can include, but are not limited to: average revenue, volume and variance in revenue and volume per service (e.g., by CPT code); average revenue, volume and variance in revenue and volume per diagnosis (e.g., by ICD code); exam order metrics per referrer or specialty; and demographic spread of payors, providers and patients. Again, such information is preferably made available to the service provider through the service provider's web account. Metrics generated by the host 150 for a payor can include, but are not limited to: average savings, cost, volume and variance in savings and cost volume per service (e.g., by CPT code); average savings, cost, volume and variance in savings and cost volume per diagnosis (e.g., by ICD code); exam order metrics per referrer or specialty; and demographic spread of payors, providers and patients. Such information is preferably made available to the payor through the payor's web account.

In accordance with an embodiment of the present invention, the company that supports the host system (referred to hereafter as the host company) can be paid service fees in various manners. For example, in one embodiment, the host company is paid a percentage of the savings that are obtained by the payor. In another embodiment, the host company is paid a percentage of the fee charged for an ordered service. Additionally or alternatively, the host company can be paid a transaction fee for each order that is processed by the host system. In one embodiment the transaction fee is paid by the payor. In another embodiment, the transaction fee is paid by the service provider that performs the ordered service. In a further embodiment, a transaction fee is paid by both the payor and the service provider. Other variations are also possible.

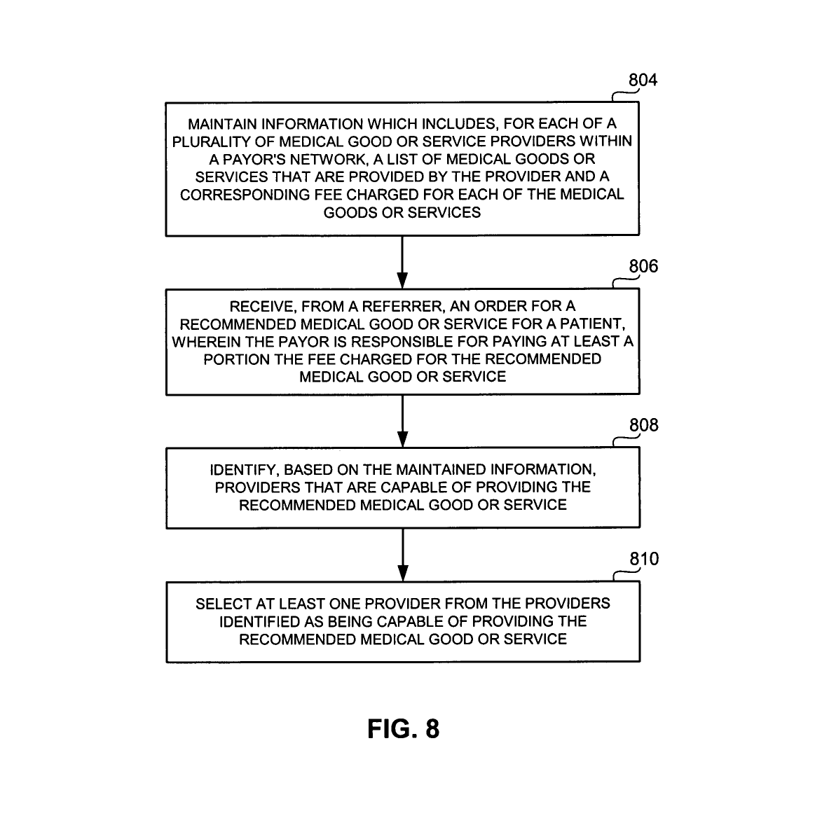

The above description of embodiments of the present invention was directed to systems and methods for reducing the costs of medical imaging services in a supply chain that includes referrers, patients, imaging service providers and payors. However, as shown in the high level flow diagram of FIG. 8, and described below, embodiments of the present invention can also be used to reduce costs for obtaining other medical services or to reduce costs for obtaining medical goods, where there is a similar supply chain.

For example, systems and methods of the present invention can also be used for lowering the costs of other types of medical services, where there is a supply chain that includes referrers (e.g., doctor's ordering other medical services), patients, other medical service providers and payors that are responsible for paying at least a portion of the fees charged by the other medical service providers. Such other medical services can include, but are not limited to, other types of medical testing, medical procedures and professional medical services. Other types of medical testing include, but are not limited to, hematological and microbiological lab testing (e.g., of samples such as blood, urine, surgical samples, etc.), ancillary medical testing (e.g., EKG, audiograms, etc.) and vision testing (e.g., eye exams). Medical procedures can include, but are not limited to, outpatient surgery (e.g., non-emergent procedures for hernia, knee, etc.), office procedures (e.g., nerve block, IV medication, colonoscopy, etc.), dental procedures (e.g., cleanings, fillings, orthodontics, etc.) and vision procedures (e.g., dilations, surgery, etc.). Professional medical services can include, but are not limited to, general medical services (e.g., exams and consultations), surgical services (e.g., exams, procedures and consultations), dental services (e.g., consultations), vision services (e.g., exams and consultations), chiropractic services (e.g., exams, adjustments and consultations), rehabilitation services (e.g., physical therapy and consultations) and podiatry (e.g., exams and consultations). Other medical services can even relate to medical staffing (e.g., physician, administration, nursing and technician staffing) and medical service support (e.g., billing, coding, etc.).

Systems and methods of the present invention can also be used for lowering the costs for obtaining medical goods, where there is a supply chain that includes referrers (e.g., doctor's ordering medical goods), patients, medical good providers and payors that are responsible for paying at least a portion of the fee charged by the medical good providers. Such medical goods can include, but are not limited to, medical supplies and medical devices. Medical supplies can include, but are not limited to, outpatient and inpatient supplies (e.g., syringes, bandages, saline, crutches, wheelchairs, etc.) and pharmaceuticals (e.g., medication). Medical devices can include, but are not limited to, surgical instruments, intravascular catheters and stents, cardiac devices (e.g., pacemakers and defibrillators), monitors, pumps, etc.

Referring to FIG. 8, step 804 includes maintaining information which includes, for each of a plurality of medical good or service providers within a payor's network, a list of medical goods or services that are provided by the provider and a corresponding fee charged for each of the medical goods or services. Still referring to FIG. 8, step 806 includes receiving, from a referrer, an order for a recommended medical good or service for a patient, wherein the payor is responsible for paying at least a portion the fee charged for the recommended medical good or service. Step 808 include identifying, based on the maintained information, providers that are capable of providing the recommended medical good or service. Step 810 includes selecting at least one provider from the providers identified as being capable of providing the recommended medical service or good. Additional details and variations of steps 804, 806, 808 and 810 can be appreciated from the above discussion of steps 704, 706, 708 and 710, respectively.

Many features of the present invention can be performed in, using, or with the assistance of hardware, software, or combinations thereof. Consequently, features of the present invention may be implemented using a processing system (e.g., including one or more processors).

Features of the present invention can be implemented in, using, or with the assistance of a computer program product which is a storage medium (media) having instructions stored thereon/in which can be used to program a processing system to perform any of the features presented herein. The storage medium can include, but is not limited to ROMs, RAMs, EPROMs, EEPROMs, DRAMs, VRAMs, flash memory devices, or any type of media or device suitable for storing instructions and/or data.

Stored on any one of the machine readable medium (media), features of the present invention can be incorporated in software for controlling the hardware of a processing system, and for enabling a processing system to interact with other mechanism utilizing the results of the present invention. Such software may include, but is not limited to, application code, device drivers, operating systems and execution environments/containers.

While various embodiments of the present invention have been described above, it should be understood that they have been presented by way of example, and not limitation. It will be apparent to persons skilled in the relevant art that various changes in form and detail can be made therein without departing from the spirit and scope of the invention.

Embodiments of the present invention have been described above with the aid of functional building blocks illustrating the performance of specified functions and relationships thereof. The boundaries of these functional building blocks have often been arbitrarily defined herein for the convenience of the description. Alternate boundaries can be defined so long as the specified functions and relationships thereof are appropriately performed. Any such alternate boundaries are thus within the scope and spirit of the claimed invention. One skilled in the art will recognize that these functional building blocks can be implemented by discrete components, application specific integrated circuits, processors executing appropriate software and the like or any combination thereof.

The breadth and scope of the present invention should not be limited by any of the above-described exemplary embodiments, but should be defined only in accordance with the following claims and their equivalents.

What is claimed is:

1. A computer implemented method for reducing costs for medical imaging services, the method comprising:
(a) maintaining service provider information which includes, for each of a plurality of medical imaging services providers within a payor's network, a list of medical imaging services that are provided by the service provider and a corresponding fee charged for each of the medical imaging services;
(b) receiving, from a referrer, an order for a recommended medical imaging service for a patient, wherein the payor is responsible for paying at least a portion of the fee charged for the recommended medical imaging service;
(c) identifying, based on the maintained service provider information, service providers that are capable of performing the recommended medical imaging service; and
(d) selecting at least one service provider capable of performing the recommended medical imaging service.

2. The method of claim 1, further comprising:
presenting, to the payor, the order for the recommended imaging service; and
allowing the payor or a designate of the payor to authorize or deny the order for the recommended imaging service.

3. The method of claim 1, wherein the selecting in step (d) is based at least in part on the fees charged by the service providers for the recommended imaging service.

4. The method of claim 1, wherein step (d) includes:
(d.1) ranking the service providers identified as being capable of performing the recommended medical imaging service, based at least in part on the fees charged by the service providers for the recommended imaging service; and
(d.2) selecting the at least one service provider based on results of the ranking.

5. The method of claim 4, wherein:
step (a) includes maintaining at least one characteristic about each of the medical imaging service providers, and maintaining a payor specified rule relating the at least one characteristic; and
step (d.1) includes ranking the service providers identified as being capable of performing the recommended medical imaging service, based at least in part on the payor specified rule relating the at least one characteristic.

6. The method of claim 4, wherein step (d.2) includes selecting only a highest ranked service provider.

7. The method of claim 4, wherein step (d.2) includes selecting at least two service providers.

8. The method of claim 7, further comprising:
(e) presenting, to the patient, the at least two service providers selected at step (d.2); and
(f) allowing the patient to select one of the at least two service providers presented.

9. The method of claim 7, wherein:
step (d) further includes identifying which one of the at least two service providers the payor would prefer perform the recommended medical imaging service; and
step (e) further includes notifying the patient of the service provider the payor would prefer perform the recommended medical imaging service.

10. The method of claim 1, wherein:
step (a) includes maintaining at least one characteristic about each of the medical imaging service providers; and
step (d) includes using one or more rule specified by the payor to filter-out one or more of the service providers identified as being capable of performing the recommended medical imaging service, wherein at least one said rule relates to a said characteristic.

11. The method of claim 10, wherein the at least one characteristic about each of the service providers includes one or more of the following characteristics:
location of the service provider;
hours of operation of the service provider; and
information about the time it takes for the service provider to complete a medical imaging service.

12. The method of claim 10, wherein the at least one characteristic about each of the service providers includes one or more of the following characteristics:
equipment of the service provider;
level of expertise of the service provider; and
patient satisfaction.

13. The method of claim 10, further comprising allowing the payor to change or add a said rule.

14. The method of claim 10, further comprising allowing the payor to specify an exception, to a said rule, which will prevent a service provider from being filtered-out even though the service provider does not satisfy said rule.

15. The method of claim 14, wherein a said exception is gradient.

16. The method of claim 15, wherein a said gradient exception to a said rule adjusts how said rule is used to filter-out one or more of the service providers identified as being capable of performing the recommended medical imaging service.

17. The method of claim 14, further comprising:
identifying a lowest fee charged for the recommended imaging service by a service provider that is capable of performing the recommended imaging service and satisfies each said rule; and
not filtering-out a service provider that is capable of performing the recommended imaging service but does not satisfy a specific said rule, if that service provider satisfies the exception to said specific rule.

18. The method of claim 14, wherein a said exception is related to a payor specified amount by which the fee, charged by a service provider that is capable of performing the recommended imaging service but does not satisfy said specific said rule, must be less than the lowest fee charged for the recommended imaging service by a service provider that is capable of performing the recommended imaging service and satisfies each said rule.

19. The method of claim 14, wherein a said exception is related to a non-monetary characteristic.

20. The method of claim 19, wherein a said exception is related to at least one of the following:
patient satisfaction;
physician satisfaction; and
hours of operation.

21. The method of claim 1, wherein:
step (a) includes maintaining location information about each of the medical imaging service providers; and
step (d) includes using the location information to filter-out one or more of the service providers identified as being capable of performing the recommended medical imaging service.

22. The method of claim 1, wherein:
step (a) includes maintaining characteristic information about each of the medical imaging service providers; and
step (d) includes using the characteristic information to filter-out one or more of the service providers identified as being capable of performing the recommended medical imaging service.

23. The method of claim 1, further comprising allowing the service providers or designate to change their corresponding service provider information, including allowing each service provider to change the fees they charge.

24. The method of claim 1, allowing the payor or designate to specify a maximum fee that can be charged for each of the medical imaging services provided by the service providers.

25. The method of claim 24, wherein fees charged by service providers for medical imaging services, included in the service provider information, can not exceed corresponding maximum fees specified by the payor.

26. The method of claim 1, wherein the list of medical imaging services included for each of the service providers includes procedure descriptors and the fee charged by the service provider for the medical imaging service corresponding to each procedure descriptor.

27. The method of claim 1, further comprising:
maintaining information that relates International Classification of Disease (ICD) numbers to procedure descriptors;
allowing a referrer to enter an ICD number;
identifying, based on the information that relates ICD numbers to procedure descriptors, one or more procedure descriptor that relates to the entered ICD number; and
notifying the referrer of the one or more procedure descriptor identified as being related to the entered ICD number, to thereby help the referrer produce the order.

28. The method of claim 27, wherein the procedure descriptors comprise Current Procedure Terminology (CPT) codes.

29. The method of claim 1, wherein the order received at step (b) includes a CPT code.

30. A machine readable medium having instructions stored thereon that when executed by one or more processor cause a system to:
have access to maintained service provider information which includes, for each of a plurality of medical imaging services providers within a payor's network, a list of medical imaging services that are provided by the service provider and a corresponding fee charged for each of the medical imaging services;
receive, from a referrer, an order for a recommended medical imaging service for a patient, wherein the payor is responsible for paying at least a portion the fee charged for the recommended medical imaging service;
identify, based on the maintained service provider information, service providers that are capable of performing the recommended medical imaging service; and
select at least one service provider from the service providers identified as being capable of performing the recommended medical imaging service.

31. A system for reducing costs for medical imaging services, the system comprising:
one or more database to maintain service provider information which includes, for each of a plurality of medical imaging services providers within the payor's network, a list of medical imaging services that are provided by the service provider and a corresponding fee charged for each of the medical imaging services;
one or more processor to
receive, from a referrer, an order for a recommended medical imaging service for a patient, wherein the payor is responsible for paying at least a portion the fee charged for the recommended medical imaging service;
identify, based on the maintained service provider information, service providers that are capable of performing the recommended medical imaging service; and
select at least one service provider from the service providers identified as being capable of performing the recommended medical imaging service.
